(19) United States
(12) Patent Application Publication
(10) Pub. No.: US 2007/0253860 A1
(43) Pub. Date: Nov. 1, 2007

(54) Process and device for sterilising ambient air

(76) Inventor: Werner Schroder, Bad Nenndorf (DE)

Correspondence Address: SHLESINGER, ARKWRIGHT & GARVEY LLP;PATENT, TRADEMARK & COPYRIGHT LAW, SUITE 600, 1420 KING STREET, ALEXANDRIA, VA, 22314, US

(21) Appl. No.: 11/785,585

(22) Filed: Apr. 18, 2007

Related U.S. Application Data
(63) Continuation of application No. PCT/EP05/11196, filed on Oct. 18, 2005.

(30) Foreign Application Priority Data
Oct. 18, 2004 (DE) 10 2004 050 657.4
Jan. 27, 2005 (DE) 10 2005 003 923.5

(51) Int. Cl. A61L 9/20

(52) U.S. Cl. 422/4; 422/121; 422/24; 422/122

(57) ABSTRACT

A process is disclosed for sterilising ambient air conducted in an air duct (401), as well as a use of a device for breaking down gaseous hydrocarbon emissions in order to sterilise ambient air conducted in an air duct (104), and a device for sterilising ambient air conducted in an air duct (401). Ambient air is supplied to the air duct (401) of an UV unit (403) for irradiation with UV radiation, and the thus pre-purified ambient air is supplied to a downstream ionization unit (407) arranged in the air duct and in which the ambient air is ionised.

### RELATED APPLICATIONS

This is a continuation application of International Application No. PCT/EP2005/011196, filed Oct. 18, 2005, which claims the priority benefit of Germany Patent Application No. DE 10 2004 050 657.4, filed Oct. 18, 2004 and Germany Patent Application No. DE 10 2005 003 923.5, filed Jan. 27, 2005, all of which are incorporated herein by reference.

### FIELD OF THE INVENTION

The invention relates to a process for sterilising ambient air conducted in an air duct, to a use of a device for breaking down gaseous hydrocarbon emissions in order to sterilise ambient air conducted in an air duct, and to a device for sterilising ambient air conducted in an air duct.

### BACKGROUND OF THE INVENTION

EP 0 778 070 B1 discloses a device for breaking down gaseous hydrocarbon emissions in an air duct, by means of which pollutant-containing exhaust air is discharged. In the known device, at least one UV emitter, which exposes the exhaust air to UV radiation having a wavelength of preferably 254 nm and a wavelength of preferably 185 nm, is provided in a first portion of the air duct, the UV radiation causing excitation of the hydrocarbons to higher energy levels and also the formation of ozone, of molecular oxygen and radicals from the ozone, and partial oxidation of the hydrocarbon molecules in the gas phase. In a subsequent second portion, there is provided a catalyst, at the surface of which catalytic oxidation of the hydrocarbon molecules is effected so that the hydrocarbon molecules are adsorbed, then oxidised on the active surface by the ozone additionally formed and/or the radicals, and are removed from the surface of the catalyst as reaction products in the form of H₂O and CO₂.

It is thus known from EP 0 778 070 B1 to convert pollutants such as solvents or odorous substances in two successive portions in an air duct conducting the ambient air. In the first portion, the reactive species required for breaking down the pollutants are produced owing to the interaction of the UV radiation and the exhaust air conducted in the air duct. The absorption of the UV light by oxygen and water molecules of the exhaust air leads to the formation of the oxidising agents ozone, hydrogen peroxide and also O and OH radicals. These have high oxidation potential and are therefore capable of oxidising pollutants. This initiates a chain reaction producing new radicals which, in turn, are able to attack other molecules. In addition, the UV radiation is absorbed by the pollutant molecules and the decomposition products thereof. As a result of the absorption of the light energy, the pollutants are excited to higher energy levels and thus activated for a reaction with the reactive species or else with atmospheric oxygen. If a sufficient amount of light energy is supplied, the molecule undergoes decomposition. The decomposition products of the photolysis of the pollutants can also form OH radicals or initiate radical chain reactions. Homogeneous gas phase reactions are started owing to the light excitation and the presence of reactive oxygen compounds. In combination with this photooxidative reaction, the first reaction stage is followed by a catalyst unit which, as the second reaction stage, allows additional degradation reactions and in which excess ozone is broken down, thus ensuring that the pollutant gas ozone does not pass into the atmosphere.

The catalyst known from EP 0 778 070 B1 is preferably an activated carbon catalyst. The activated carbon used is a highly porous material having an internal surface area of approx. 1,200 m²/g which is used as a reaction surface. The purpose of the activated carbon is firstly to retain compounds which are difficult to oxidise, thus increasing their residence time in the reactor. This increases the concentration of these components compared to the gas phase, leading to a rise in the speed of reaction with the formed oxygen species on the surface of the activated carbon. Secondly, the use of the activated carbon as a downstream catalyst ensures that the pollutant ozone does not pass into the environment, as activated carbon acts as an ozone filter.

EP 0 778 070 B1 also mentions providing ionisation of the exhaust air in a third portion.

The device known from EP 0 778 070 B1 and the process known therefrom are used for breaking down odorous substances and pollutants contained in the exhaust air, in particular in the form of hydrocarbons. Other uses of this device and this process are not known.

U.S. Pat. No. 5,230,220 discloses an air purification device for the interior of a refrigerator used, inter alia, for the reduction of bacteria in the air supplied to the air purification device. The air purification device comprises a UV emitter and also a catalyst, the air to be purified firstly passing through the UV emitter and then flowing through the catalyst. The purpose of the catalyst is to break down the excess ozone produced by the UV emitter.

WO 91/00708 A1 describes a compact air purification device integrated in a lamp socket. In the interior of the lamp socket, there is a UV emitter around which a filament is wound. The filament is intended to produce heat inside the lamp socket and at the same time ionise the air located in the lamp socket. An integrated fan draws in air through the base of the lamp socket. A filter, through which the drawn-in air leaves the lamp socket again, is located at the upper edge of the of the lamp socket. The UV emitter and filament act on the air flowing by as a common reaction stage. Reference is made to the fact that this air purification device can also be used for killing off microorganisms.

JP 062 05930 A discloses a device and a process for purifying ambient air contaminated with cigarette smoke. One embodiment shows a UV emitter around which the electrode of an ionisation unit is wound. In this embodiment, the UV emitter and ionisation unit also act on the air flowing by as a common reaction stage.

A drawback of the known devices and processes is the restricted field of application. For example, the operation of air-conditioning systems displayed the need to sterilise the air circulated in the air-conditioning system. On account of their low throughputs, in particular, the known devices and processes are not suitable for a field of application of this type. The device known from EP 0 778 070 B1 presupposes the presence of hydrocarbons.

### OBJECTS AND SUMMARY OF THE INVENTION

The object of the invention is therefore to find a device and a process for sterilising ambient air conducted in an air duct.

This object is achieved by a process according to Claim 1, a use of a device according to Claim 5, and a device according to Claim 22.

The basis of the invention and, in particular, of the method according to the invention in accordance with Claim 1 is, in this regard, the connection of the UV unit and ionisation unit. It has been found that a highly effective sterilising effect of the ambient air supplied to the air duct and, at the same time, long-lasting sterilisation of the ambient air discharged from the air duct occur if the air duct consists of a UV unit and a subsequent ionisation unit.

The UV unit causes a killing-off of microorganisms based substantially on the formation of reactive reaction agents such as ozone and/or oxygen radicals and also on the absorption of the UV radiation.

It is known that the formation of reactive reaction agents such as ozone and/or oxygen radicals, and thus an ozone-producing effect, can be achieved, in particular, if the wavelength of the radiation emitted by each UV unit is below 240 nm, for example in the region of 185 nm. Owing to the formation of ozone, the sterilising effect occurs in the wavelength range below 240 nm, in particular, as a result of the oxidation of the microorganisms.

Moreover, absorption of the UV radiation by the microorganisms and also the formation of radicals by UV radiation above 240 nm, for example in the region of 254 nm, can be achieved. Killing-off of the microorganisms can initially be achieved in that the UV radiation is absorbed by the microorganisms. In this wavelength range, the already produced ozone is also cleaved back into an oxygen molecule and a reactive oxygen atom, so the above-described sterilising effect resulting from radicals also occurs in this wavelength range. Finally, the radiation emitted in this range causes the excitation of the organic molecules contained in the ambient air, such as for example hydrocarbons, to higher energy levels. This also provides a sterilising effect as a result of the killing-off of the microorganisms contained in the ambient air.

The ambient air pre-purified in this form is supplied in the air duct to an ionisation unit which follows the UV unit and in which the ambient air is ionised. A preferred embodiment provides for the ionisation unit to consist of at least one ionisation tube. In an ionisation tube, two electrodes are separated from each other by a non-conductive dielectric. The ionisation is based in this case on a controlled discharge of gas which occurs between the two electrodes and the dielectric located therebetween, the electrodes typically being activated with an AC voltage having peak values of between 500 V and 10 kV. The frequency of the AC voltage is preferably in the region of 50 Hz, although high-frequency AC voltages of up to 50 kHz can also be used. The gas discharge is a barrier discharge, the dielectric acting as a dielectric barrier. This produces time-limited individual discharges preferably distributed homogeneously over the entire electrode surface. It is characteristic of these barrier discharges that the transition into a thermal arc discharge is prevented by the dielectric barrier. The discharge breaks off before the high-energy electrons (1 to 10 eV) resulting during the ignition discharge their energy to the surrounding gas by thermalisation. The energy released by the discharge process is taken up by the oxygen and hydrogen molecules in the air, oxygen and hydroxyl radicals and also oxygen ions and ozone molecules being formed. On account of their high energy and charge state, these species are chemically highly reactive and seek to combine with oxidisable substances such as organic and inorganic odorous substances. This chemically changes the odorous substances, so new, non-odorous and innocuous substances (for example H₂O and CO₂) are formed. In addition, the reactive species are also capable of harming and killing off the microorganisms still remaining from the first two reaction stages.

The ions produced in the ionisation unit can have a residence time of a few hours. A further effect of the ionisation is therefore that the produced ions are further conveyed by the ambient air conducted in the air duct and can also still achieve a purifying effect in the subsequent units.

Nevertheless, it should be noted that if merely a UV unit is used in combination with an ionisation unit, the sterilised air can have a high ozone content after leaving the device. A sterilising device of this type is therefore restricted to areas in which the produced ozone cannot exert a harmful effect.

Although it is in principle possible, for breaking down ozone, to arrange a catalyst after the ionisation unit, this again has the drawback that the ions produced by the ionisation unit are typically also neutralised in the catalyst, thus reducing again the purifying effect of the ions in downstream portions. In order nevertheless to achieve a desired amount of ions in the air leaving the catalyst, use would have to be made of a catalyst material which either selectively catalyses the breaking-down of ozone or at least promotes it over the breaking-down of ions.

A further solution according to the invention in accordance with Claim 5 therefore consists in using a device known per se for breaking down gaseous hydrocarbon emissions now for sterilising ambient air conducted in an air duct.

In a device of this type, there are provided in a first portion of the air duct a UV unit for irradiating the ambient air with the UV radiation, in a subsequent second portion a catalyst for breaking down the ozone produced by the UV unit, and in a subsequent third portion an ionisation unit for ionising the ambient air.

A fundamental finding of this solution according to the invention therefore consists in the fact that the device known per se for breaking down hydrocarbon emissions exerts a sterilising effect on ambient air, the presence of hydrocarbon emissions in the ambient air no longer having to be a prerequisite for achieving the sterilising effect. In the past, it was assumed that a device of this type can be used merely for breaking down pollutants of hydrocarbon emissions.

A further solution according to the invention consists, according to Claim 22, of a device known per se comprising a UV unit for irradiating the ambient air with UV radiation in a first portion of the air duct, comprising a catalyst for breaking down the ozone produced by the UV unit in a subsequent second portion and comprising an ionisation unit for ionising the ambient air in a subsequent third portion. This finding according to the invention in accordance with this solution according to the invention consists in providing a filter for microorganisms between the first portion and the second portion, as a result of which the device is able to sterilise the ambient air conducted in the air duct.

In accordance with this solution according to the invention, the microorganisms are therefore held off by the filter and are thus unable to pass into the catalyst. Preferably, the filter is arranged in this case so close to the UV tubes that the microorganisms are effectively killed off owing to the long-term irradiation.

Preferred embodiments of the solutions according to the invention will be described hereinafter.

A preferred embodiment provides for the UV unit to consist of at least one cylindrically configured UV emitter. The aforementioned wavelength ranges of 185 nm and 254 nm can be produced, for example, using mercury vapour lamps. In order to be able to cover the aforementioned wavelength ranges and, in particular, the range below 240 nm, when using conventional mercury vapour lamps, it is necessary in this regard for the glass type of the glass surrounding the mercury vapour lamp not to absorb these wavelength ranges. This requirement can be met, for example, by synthetic quartzes.

According to a further preferred embodiment, provision is made for the first portion of the air duct to have reflective surfaces in the region of the UV radiation. This allows the intensity of the UV radiation to be amplified.

According to a further preferred embodiment, provision is made for the inner walls of the air duct to have, in the region of the UV radiation, a coating for achieving a photocatalytic effect. A photocatalytic effect can, for example, be achieved by the coating comprising a broadband semiconductor material and has already been described in WO 2005/002638 A2 and DE 103 30 114 A1. It has been found that titanium dioxide (TiO₂) or doped titanium dioxide is especially suitable as a semiconductor material. As a result of the irradiation of the titanium dioxide or doped titanium dioxide with UV radiation, the energy of which is greater than or equal to the difference in energy between the valence band and conduction band of the semiconductor, electron/hole pairs are initially generated in the semiconductor material. There are then formed oxygen-containing radicals which effectively assist the process of the oxidation of microorganisms and therefore the killing-off of microorganisms. The sterilising effect of this photocatalytic process thus occurs, in particular, on the coated surfaces themselves, thus allowing a further rise in the efficiency of the sterilising device to be achieved.

In addition, it has been found that the distance between the UV emitter and the inner walls of the air duct is to be taken into account for achieving optimum interaction between the UV radiation and the catalyst material. For optimising an air duct of this type, the distance is therefore always chosen in such a way that, for a given catalyst material and predetermined UV emitter, an optimum rate of decomposition of the respective pollutants can be achieved.

This photocatalytic effect can, in principle, be achieved over the entire wavelength range of the described UV emitters. Tests using titanium dioxide have revealed that an especially marked photocatalytic effect occurs at a wavelength of the radiation emitted by each UV emitter in the range of between 350 nm and 420 nm.

The catalyst used preferably consists of an activated carbon filter. The basic construction of the activated carbon filter consists in this case of a container which is filled with activated carbon and through which the ambient air is conducted.

Also possible is the use of what are known as support catalysts which are composed of a support material, known as the skeleton substance, and certain additives, known as promoters. Activated carbon, pumice stone, zeolites or clay can, for example, be used as support materials. The additives may be catalytically active metal oxides, in particular oxides of Mn, Fe, Co, Ni, Zn, Si, Ti or Zr. It is also possible, within the scope of the invention, to use the noble metals Pt, Pd or Rh as additives.

Optionally, it is also possible for the additives to consist of mixtures of the aforementioned metal oxides and the aforementioned noble metals. Known methods for producing the support catalyst include, for example, precipitation and impregnation. In the former method, the active components are precipitated from the corresponding saline solutions. The impregnation method is based on a saturation of the support material with metal saline solutions or melts (for example metal oxide melts) and by the application of the active components to the support from the vapour phase.

According to a further preferred embodiment, a zigzag arrangement of the catalyst container allows the wall thickness thereof, and thus also the flow resistance thereof, to be reduced at a predetermined volume.

It has been found that the devices on which the solutions according to the invention are based can be used effectively in ventilation systems in order lastingly to sterilise the ambient air conducted therein, as the air flow rate required for this purpose can be achieved. For conventional commercial air-conditioning systems, provision is made, for example, for the ambient air filling the room to be ventilated to be circulated several times per hour.

The sterilisation according to the invention of the ambient air conducted in the air duct includes, in this case, the killing-off of the microorganisms contained in the ambient air to a degree compatible with human health. The microorganisms to be killed off include viruses, bacteria, yeasts or else fungal spores. It was found that ambient air contaminated even with enveloped viruses can, in particular, be effectively sterilised. This applies, inter alia, to SARS viruses, avian flu viruses, Ebola viruses and influenza viruses.

### BRIEF DESCRIPTION OF THE DRAWINGS

The invention will be described hereinafter in greater detail on the basis of various embodiments with reference to the enclosed drawings, in which:

FIG. 1 is a block diagram concerning the arrangement of the basic device comprising two portions,

FIG. 2 is a cross section of an air duct with the arrangement of the basic device comprising two portions according to a first embodiment,

FIG. 3 is a block diagram concerning the arrangement of a device comprising three portions,

FIG. 4 is a cross section of an air duct with the arrangement of three portions according to a second embodiment,

FIG. 5 is a cross section of an air duct with the arrangement of three portions according to a third embodiment,

FIG. 6 is a block diagram in which the sterilising system according to the invention is connected in an air-conditioning system,

FIG. 7 is a perspective view of three portions connected in series according to a fourth embodiment,

FIG. 8 is a perspective view of a purifying system comprising three portions according to the fourth embodiment from FIG. 7,

FIG. 9 is a perspective view of three portions connected in series according to a fifth embodiment,

FIG. 10 is a perspective view of a purifying system comprising three portions according to the fifth embodiment from FIG. 9,

FIG. 11 is a perspective view of a purifying device according to a sixth embodiment,

FIG. 12 is a cross section of a purifying device according to the sixth embodiment,

FIG. 13 is a cross section of a purifying device according to a seventh embodiment,

FIG. 14 is a cross section of a purifying device according to an eighth embodiment, and

FIG. 15 is a cross section of a purifying device according to a ninth embodiment.

### DETAILED DESCRIPTION OF THE INVENTION

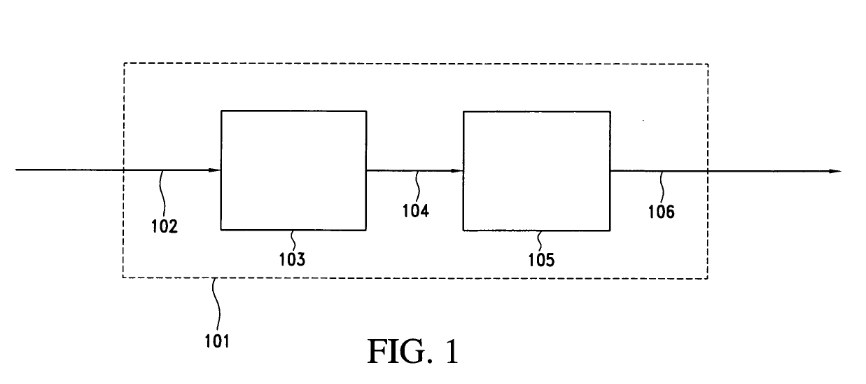

FIG. 1 is a block diagram concerning the arrangement of the basic device comprising two portions. The first portion contains the UV unit, whereas the second portion contains the ionisation unit. The two portions form as a unit a purification stage 101 which is integrated into the air duct of a ventilation system. However, it should be noted that the air 106 issuing from the purification stage 101 has a high ozone content and precautions therefore have to be taken to neutralise the ozone before the sterilised and purified air flows into the room to be ventilated. In the operation of air-conditioning systems, in particular, the problem repeatedly occurs that there can multiply within the air-conditioning system harmful microorganisms such as viruses, fungal spores, yeasts and bacteria which can then lead to an adverse health effect in the ventilation of rooms. The purification stage 101 is thus preferably connected to an air duct conducting the respective ambient air, so the ambient air in the air duct can be conveyed from one reaction stage to the following reaction stage.

The ambient air 102 entering the purification stage 101 is supplied to the first portion 103 containing a UV unit for irradiating the passing ambient air with UV radiation. The microorganisms contained in the ambient air are effectively killed off by the UV radiation. In addition, the UV irradiation also causes the formation of ozone, of molecular oxygen and of radicals from the ozone. The ambient air 104 pre-treated in this form is then supplied to the second portion 105 which has an ionisation unit for ionising the ambient air. The ionisation produces additional oxygen and hydroxyl radicals and also oxygen ions and ozone molecules which, on account of their high energy and charge state, seek to combine with oxidisable substances. This chemically changes organic and inorganic odorous substances, so new, non-odorous and innocuous substances (for example H₂O and CO₂) are formed. In addition, the ionisation of the air has an additional germ-killing effect, so the air 106 issuing from the second reaction stage can be fed back as sterilised air to a subsequent ventilation portion.

Nevertheless, on account of the high reactivity of the two reaction stages 103 and 105, it should be noted that the issuing air 106 has, directly at the output of the second reaction stage 105, an ozone content which can exceed the admissible limits for the ventilation of rooms. However, this effect can successfully be utilised in that the purification stage 101 precedes, for example, the central device, located in the air duct, of an air-conditioning system. The purified ambient air 106 loaded with ozone and ions can in this way initially pass through the central device of the air-conditioning system and thus also produce a purifying and sterilising effect within the central device of the air-conditioning system.

If the ambient air supplied to the room still has an excessively high concentration of ozone, a catalyst can be provided to break down the ozone contained in the supplied ambient air to an admissible degree. However, it should be noted in this regard that the catalyst can also inhibit the above-mentioned further conveyance of the ions produced in the second reaction stage. In order nevertheless to achieve a desired amount of ions in the air leaving the catalyst, use must be made of a catalyst material which either selectively catalyses the breaking-down of ozone or promotes it over the breaking-down of ions. Alternatively, in this case, a second ionisation unit can also follow the catalyst, again allowing the generation of ions which can produce a purifying effect in subsequent portions or the room itself to be ventilated.

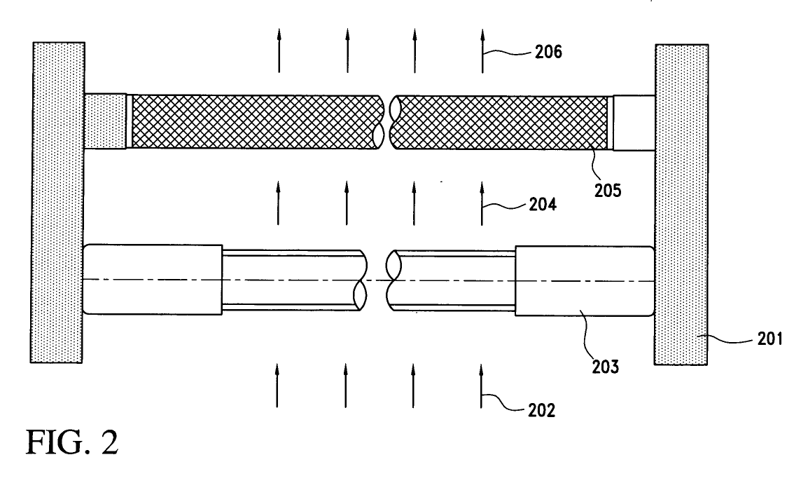

FIG. 2 is a cross section of an air duct with the arrangement of the basic device comprising two portions according to a first embodiment. A UV tube 203 and an ionisation tube 205 are connected directly between the walls of the air duct 201. The entering ambient air 202 initially flows around one or more UV tubes 203. The ambient air 204 thus pre-treated then flows around one or more ionisation tubes 205 before the air 206 then issuing can be further conveyed as purified and sterilised air in the air duct 201. This design according to the first embodiment can be kept very compact and therefore easily integrated into existing systems. A device according to this embodiment can also be used for sterilising, for example, surfaces contaminated with SARS viruses. Experimental tests carried out on a cell culture infected with SARS viruses revealed that an arrangement according to FIG. 2, with a distance of approximately 20 cm between the ionisation unit and the surface to be sterilised and a distance of approximately 3 cm between the UV unit and the surface to be sterilised, led to rapid killing-off of the SARS viruses located on the surface within a cell culture. Owing to empirical considerations, the experiment was carried out using a natural air stream. However, it was found in this case that this natural air stream is sufficient in the sterilising of surfaces contaminated with viruses and an air flow through an air duct does not have to be generated. Samples were taken from two respective depressions, at the start and several times over a period of 40 minutes, from a cell culture exposed to the sterilising device and from a control cell culture plate which was not exposed to UV radiation and ionised air. Double samples were taken in each case and stored under cool conditions. 55 μl of all samples were then transferred to 96-well cell culture plates and dilution series were applied to base 10 (10⁰ to 10⁻⁷) in quadruple analysis. These dilutions were mixed with trypsinised vero cells and incubated for 4 days in a cell culture incubator at 37° C. in the presence of 5% CO₂. The state of the cells was checked daily using a microscope. After completion of the experiment after four days, it was found that the treatment using the sterilising device drastically reduced the infectivity of the SARS viruses. The infectivity of the SARS viruses could be reduced to a level below the detection limit after treatment using this device for just 1 minute. The samples obtained after sterilising for 20 minutes contained a substance which, at a highest concentration (10⁰), had a toxic effect on the cell culture. This effect also occurred during sterilising for 30 and 40 minutes. Compared to data in the specialist literature (Duan et al., Stability of SARS coronavirus in human specimens and environment and its sensitivity to heating and UV irradiation, SARS Research Team, Biomed. Environ. Sci. September 2003 16(3): 246 to 255), according to which the infectivity of SARS viruses is inactivated after irradiation for 1 hour with UV light, the tested sterilising system demonstrated, as a result of inactivation, significant acceleration of the sterilising process after as little as 1 minute.

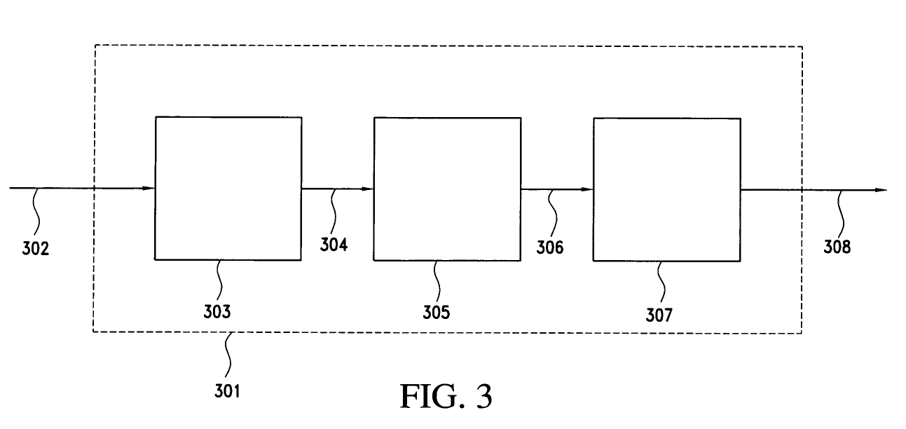

FIG. 3 is a block diagram concerning the arrangement of the device comprising three portions. Basically, the three portions form a sterilising system 301 integrated into the air duct of a ventilation system.

The basic construction of the sterilising system 301 consists of a first portion 303, a second portion 305 and a third portion 307.

The ambient air 302 entering the sterilising system 301 is supplied to the first portion 303 containing a UV unit for irradiating the passing ambient air with UV radiation. The ambient air 304 thus pre-treated is then supplied to the second portion 305 in which excess ozone on the surface of the catalyst is broken down to form molecular oxygen. The ozone generated in the first portion therefore does not have any harmful effect on the environment. The ambient air 306 present on leaving the second portion is then supplied to the third portion 307 which has an ionisation unit for ionising the ambient air. The purified air 308 leaves the sterilising system 301.

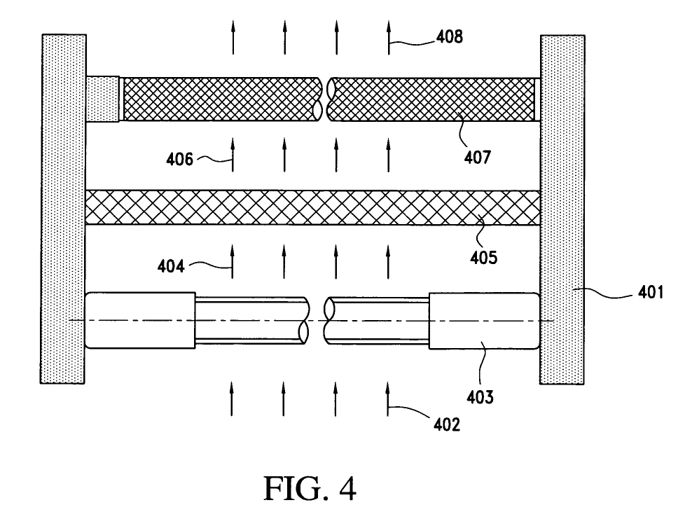

FIG. 4 is a cross section of an air duct with the arrangement of three portions according to a second embodiment. A UV tube 403, a catalyst 405 and an ionisation tube 407 are connected directly between the walls of the air duct 401. The entering ambient air 402 initially flows around one or more UV tubes 403. The ambient air 404 thus pre-treated then flows through the catalyst 405. Finally, the ambient air 406 thus further treated flows around one or more ionisation tubes 407 before the ambient air 408 then issuing can be further conveyed as purified and sterilised air in the air duct 401.

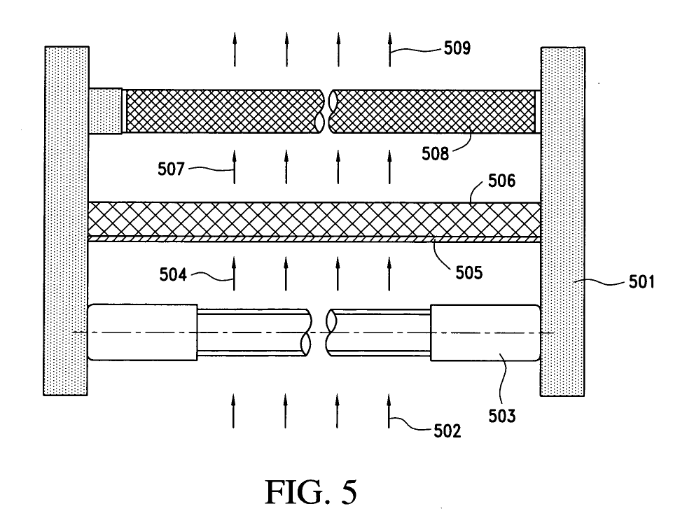

FIG. 5 is a cross section of an air duct with the arrangement of three portions according to a third embodiment. A UV tube 503, a catalyst 506 comprising a filter 505 for microorganisms and an ionisation tube 508 are connected directly between the walls of the air duct 501. The entering ambient air 502 flows initially around one or more UV tubes 503. The ambient air 504 thus pre-treated then flows through the filter 505 and the catalyst 506. The filter 505 holds off the microorganisms still contained in the ambient air 504, an additional sterilising effect being achieved as a result of the continuous irradiation of the filter by the UV tubes. Finally, the ambient air 507 thus further treated flows around one or more ionisation tubes 508 before the ambient air 509 then issuing can be further conveyed as purified and sterilised air in the air duct 201.

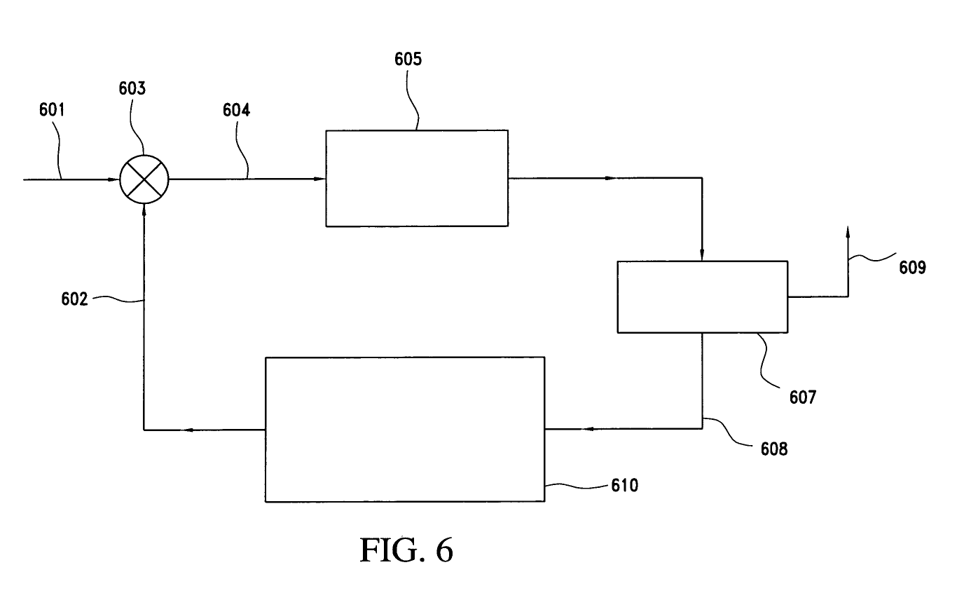

FIG. 6 is a block diagram in which the sterilising system according to the invention is connected in an air-conditioning system. The illustrated system consists of an air mixer 603, a sterilising system 605, a central device of the air-conditioning system 607 and also the room 610 filled with ambient air. Microorganisms are intended to be prevented from multiplying in the central device of the air-conditioning system 607. For this purpose, the sterilising system 605 precedes the central device of the air-conditioning system 607.

Supplied fresh air 601 is initially mixed with the outgoing air 602 of the room 610 in the air mixer 603. The air 604 thus mixed is supplied to the sterilising system 605. The sterilising system 605 consists in this case of one of the above-described connections in series of a plurality of portions according to the first, second or third embodiment. For example, the sterilising system 605 can consist of a first portion comprising a UV unit, a second portion comprising a catalyst and an upstream filter for microorganisms, and a third portion comprising an ionisation unit. The air 608 brought to the desired temperature is then fed back to the room 610. The drop in temperature generated by the central device of the air-conditioning system 607 is transferred to the air 609 and removed.

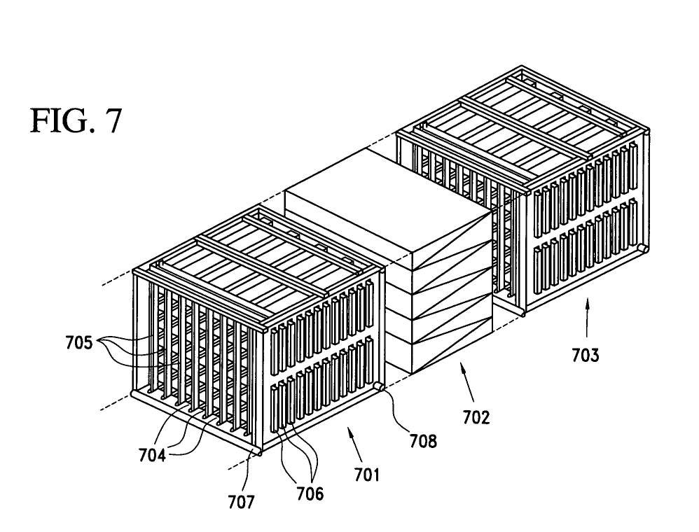

However, for high volume flow rates, it has also proven beneficial to arrange the UV emitters and ionisation tubes shown in FIG. 2, FIG. 4 and FIG. 5 not transversely but rather longitudinally to the air stream. FIG. 7 is a is perspective view of three portions 701, 702, 703 connected in series according to a fourth embodiment which provides for the UV emitters and ionisation tubes to be arranged longitudinally to the air stream. The three portions 701, 702, 703 are designed as box-type inserts which can be inserted into a rectangular air duct. The first portion comprises a large number of honeycomb reaction channels 704 connected in parallel. A UV emitter is arranged longitudinally in each of the reaction channels of the first portion. The first portion is followed by the second portion containing the catalyst 702. The catalyst can, for example, consist of activated carbon material as described hereinbefore. In the illustrated embodiment, the catalyst consists of a thin-walled construction fitted into the air duct in a zigzag configuration. A filter for microorganisms can precede the catalyst 702. The third portion 703 comprises, in turn, a large number of honeycomb reaction channels which are connected in parallel and in each of which an ionisation tube is longitudinally arranged.

For the sake of simplicity, the construction of the first portion 701 comprising the UV emitters contained therein will be described hereinafter. The similar construction applies accordingly to the third portion 703 comprising the ionisation tubes contained therein.

A respective tubular UV emitter is arranged in each reaction channel 704 of the first portion 701. The reaction channels 704 interconnected in this way are surrounded by a metal housing. Provided at the air inlet opening and the air outlet opening are respective contact rails 705 which firstly act as cable channels for the electrical feeds to the UV emitters and which secondly mechanically hold the UV emitters in the reaction channels 704. Laterally corresponding power supply units 706 are provided for electrically activating the UV emitters. Slide rails 707 and 708 are provided on the undersides of the first portion 701 to allow the first portion 701 in the air duct to be inserted or removed on corresponding rollers for maintenance purposes.

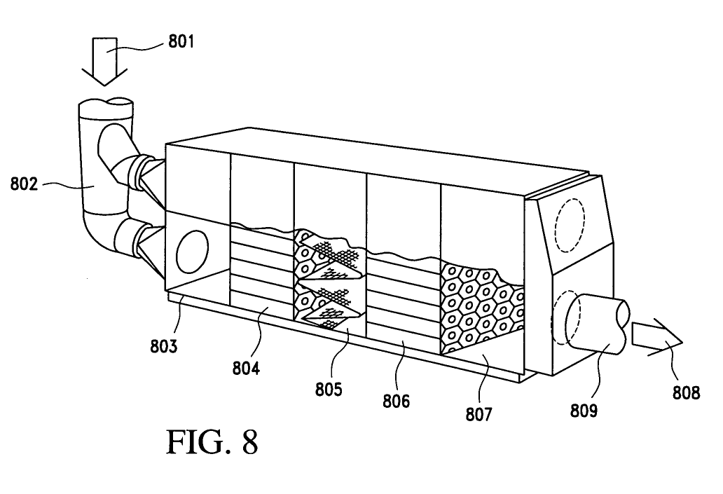

FIG. 8 is a perspective view of a purifying system comprising three portions according to the fourth embodiment from FIG. 7. The ambient air 801 contaminated with pollutants passes initially into a distributor chamber 803, in which the supplied air is distributed uniformly, via a supply pipe 802. The distributor chamber is followed by a first portion 804, a second portion 805 and a third portion 806 which correspond, in terms of their construction, to the three portions 701, 702 and 703 according to FIG. 7, so reference is made in this case to the foregoing description of FIG. 7. The second portion 805 directly follows the first portion 804 and the third portion 806 directly follows the second portion 805. The third portion 806 is followed by a further distributor chamber 807 before the ambient air 808 thus purified and sterilised is further conducted via a discharge pipe 809. There is preferably located in the course of the discharge pipe 809 a suction fan which ensures the conveyance of the ambient air, as in this way only the already purified and sterilised ambient air 808 passes through the suction fan.

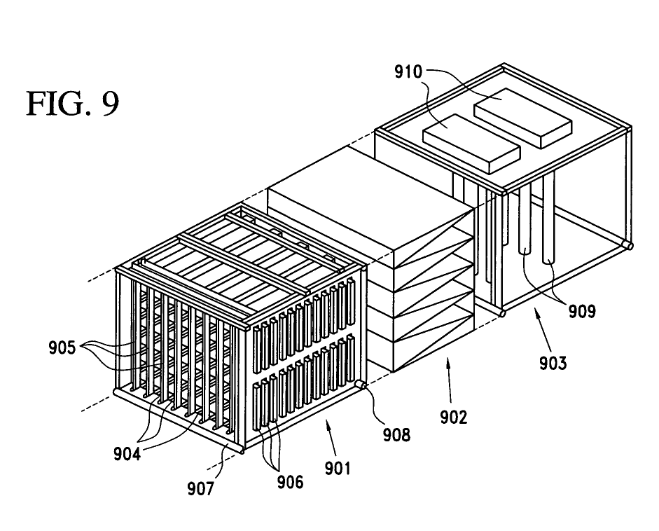

FIG. 9 is a perspective view of three portions 901, 902, 903 connected in series according to a fifth embodiment which provides for the UV emitters to be provided longitudinally to the air stream and the ionisation tubes to be arranged perpendicularly to the air stream. The three portions 901, 902, 903 are designed as box-type inserts which can be inserted into a rectangular air duct. The first portion comprises a large number of honeycomb reaction channels 904 connected in parallel. A UV emitter is arranged longitudinally in each of the reaction channels of the first portion. The first portion is followed by the second portion comprising a catalyst 902. The catalyst can, for example, consist of activated carbon material as described hereinbefore. In the illustrated embodiment, the catalyst consists of a thin-walled construction which is fitted into the air duct in a zigzag configuration. A construction of this type can also be chosen for the combined catalyst and a filter for microorganisms preceding it. The third portion 903 comprises a large number of ionisation tubes arranged perpendicularly to the direction of flow.

The construction of the first portion 901 comprising the UV emitters contained therein corresponds to that of the first portion 701 from FIG. 7, so reference is made to the corresponding description of FIG. 7.

The ionisation tubes 909 of the third portion 903 are fastened to what are known as insert devices 910 and installed perpendicularly to the direction of flow. Each insert device comprises in this case a specific number of ionisation tubes. The total number of the ionisation tubes 909 and the size thereof are chosen as a function of the three-dimensional configuration and also the specific atmospheric loads. The insert devices 910 can in this case comprise an intensity regulator by means of which the tube tension can be set as required. It is, however, also possible automatically to regulate the intensity of the ionisation tubes 909 using a gas sensor. The regulation can, for example, be carried out using a gas sensor as is described according to WO 2004/014442 A1 or DE 102 36 196 A1. The compensation regulation described in said documents ensures that air can be purified as required even in the case of extreme and/or rapidly alternating atmospheric loads.

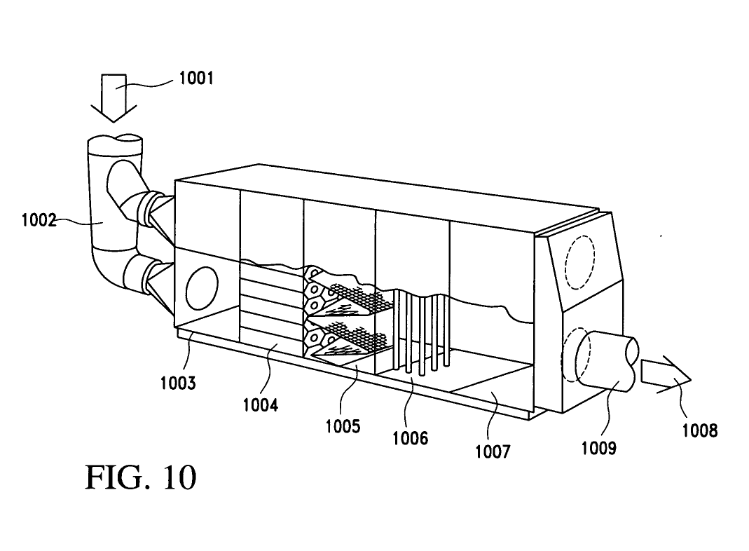

FIG. 10 is a perspective view of a purifying system comprising three portions according to the fifth embodiment from FIG. 9. The ambient air 1001 contaminated with pollutants passes initially into a distributor chamber 1003, in which the supplied air is distributed uniformly, via a supply pipe 1002. The distributor chamber is followed by a first portion 1004, a second portion 1005 and a third portion 1006 which correspond, in terms of their construction, to the three portions 901, 902 and 903 from FIG. 9, so reference is made in this case to the description of FIG. 9. The second portion 1005 directly follows the first portion 1004 and the third portion 1006 directly follows the second portion 1005. The third portion 1006 is followed by a further distributor chamber 1007 before the ambient air 1008 thus purified and sterilised is further conducted via a discharge pipe 1009. There is preferably located in the course of the discharge pipe 1009 a suction fan which ensures the conveyance of the ambient air, as in this way only the already purified and sterilised ambient air 1008 passes through the suction fan.

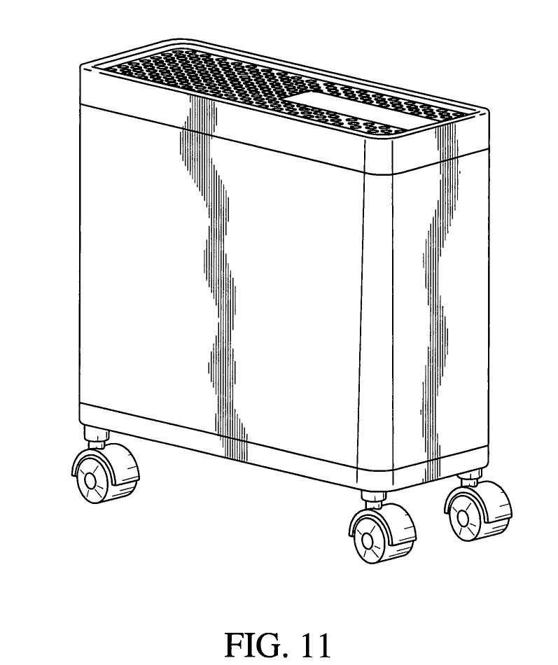

FIG. 11 shows a purifying device according to a sixth embodiment. This system is relatively compact compared to the fourth and fifth embodiments and does not have to be integrated into an air-conditioning system and can accordingly be operated as a free-standing device. The fields of application include in this case, inter alia, doctors' practices, rooms in hospitals such as, for example, a sick room, nurseries or consultation rooms. The device is operated using a conventional supply terminal, transformers, power supply units and any control means being accommodated in a region of the housing shown in FIG. 11. Depending on the field of application, the purifying device can either be equipped with rollers, as illustrated in FIG. 11, or stand on fixed feet.

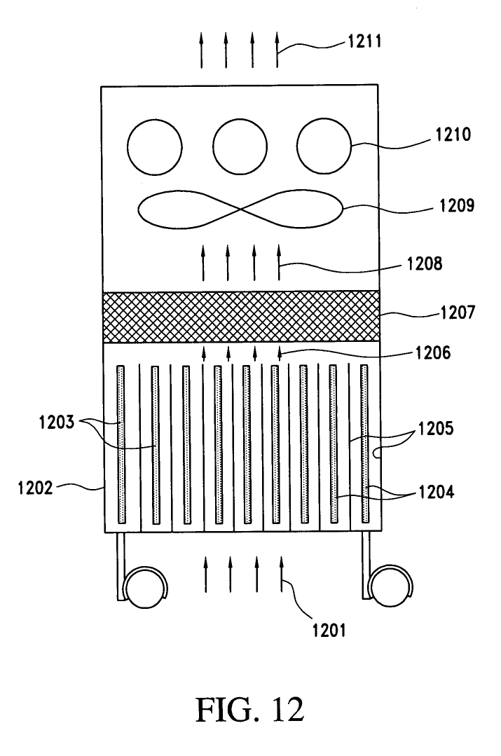

FIG. 12 is a cross section of a purifying device according to the sixth embodiment. It is preferably designed for movable use, for example for the purifying and sterilising of air in aircraft on the ground during maintenance work, in ships or hospitals. The ambient air 1201 contaminated with pollutants passes into the purifying device via inlet openings on the underside of the housing 1202. The ambient air 1201 contaminated with pollutants passes in this case initially through a first portion. The first portion comprises a large number of reaction channels 1203 arranged in a honeycomb configuration and connected in parallel. A UV tube 1204 is arranged longitudinally in each of the reaction channels 1203 of the first portion. The walls 1205 of the reaction channels 1203 are preferably coated with a reflective material. The arrangement of the UV tubes 1204 in the direction of flow allows the purifying device to be operated at high volume flow rates. The air 1206 pre-treated in this way then passes through the second portion consisting of a catalyst 1207. The air 1208 issuing from the second portion then passes into the suction fan 1209 which ensures that the air is conveyed through the purifying device. Finally, the air passes through a third portion consisting of ionisation tubes 1210. The ionisation tubes are preferably arranged perpendicularly to the direction of flow to allow a low overall height of the purifying device. The purified air 1211 issues through openings on the upper side of the housing 1202.

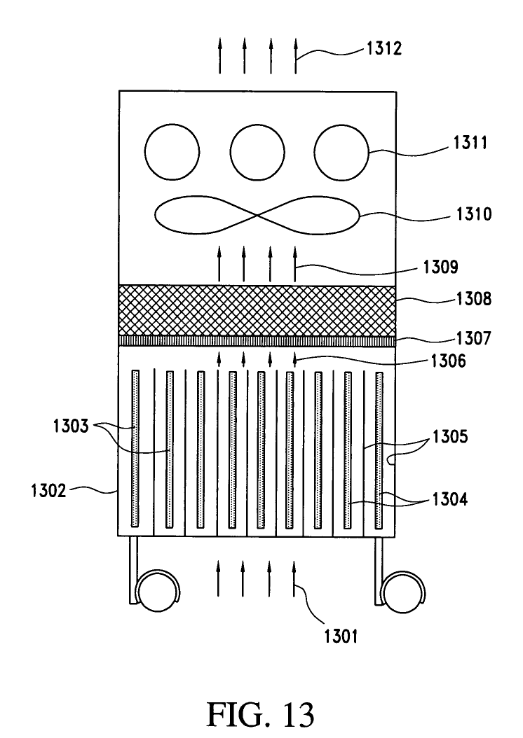

FIG. 13 is a cross section of a purifying device according to a seventh embodiment. Like the sixth embodiment, it is preferably designed for movable use and can be accommodated in a corresponding housing, for example according to FIG. 11. The ambient air 1301 contaminated with pollutants passes into the purifying device via inlet openings on the underside of the housing 1302. The ambient air 1301 contaminated with pollutants passes in this case initially through a first portion. The first portion comprises a large number of reaction channels 1303 which are arranged in a honeycomb configuration and connected in parallel. A UV tube 1304 is arranged longitudinally in each of the reaction channels 1303 of the first portion. The walls 1305 of the reaction channels 1303 are preferably coated with a reflective material. The arrangement of the UV tubes 1304 in the direction of flow allows the purifying device to be operated at high volume flow rates.

The air 1306 pre-treated in this way then passes through the second portion consisting of a filter for microorganisms 1307 and a subsequent catalyst 1308. The air 1309 issuing from the second portion then passes into the suction fan 1310 which ensures that the air is conveyed through the purifying device. Finally, the air passes through a third portion consisting of ionisation tubes 1311. The ionisation tubes are preferably arranged perpendicularly to the direction of flow to allow a low overall height of the purifying device. The purified air 1312 issues through openings on the upper side of the housing 1302.

A drawback of this embodiment is that the filter for microorganisms 1307 is irradiated by the UV tubes 1304 only to a limited extent. The killing-off of microorganisms trapped by the filter for microorganisms 1307 is therefore not as effective as in the third embodiment according to FIG. 5. A further drawback is that large particles of dirt can also advance up to the filter for microorganisms 1307. In the event of excessive contamination, the filter for microorganisms 1307 therefore has to be exchanged.

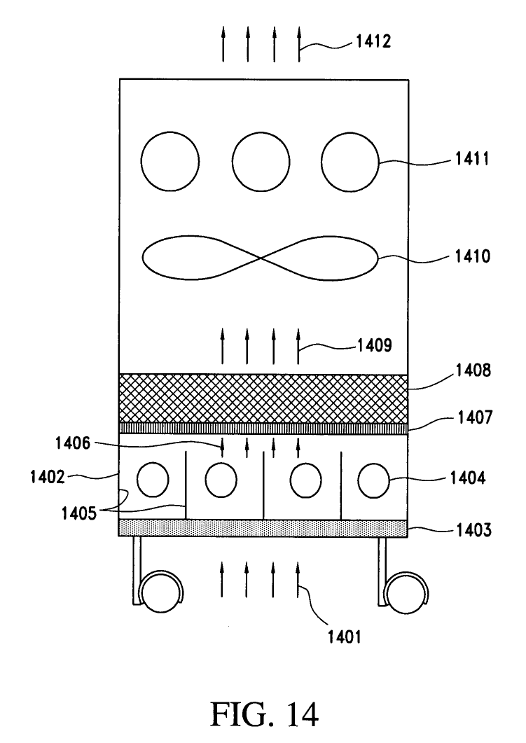

FIG. 14 is a cross section of a purifying device according to an eighth embodiment. The ambient air 1401 contaminated with pollutants passes into the purifying device via inlet openings on the underside of the housing 1402. Firstly, the ambient air 1401 contaminated with pollutants passes through a dust filter 1403. On the one hand, this traps large particles of dirt such as grains of dust; on the other hand, some microorganisms also become stuck in the dust filter 1403. These microorganisms are rendered harmless by the continuous UV irradiation of the subsequent UV tubes 1404. The air passed through the dust filter 1403 then passes through the first portion consisting of the UV tubes 1404 and reflective surfaces 1405. The UV tubes 1404 are in this case preferably arranged perpendicularly to the direction of air flow to allow a low overall height of the purifying device. At the same time, this arrangement provides optimum irradiation of the dust filter 1403, allowing effective killing-off of trapped microorganisms. The reflective surfaces 1405, which are located between the UV tubes 1404 and on the lateral walls of the housing 1402, intensify the effect of the UV radiation. The air 1406 pre-treated in this way then passes through the second portion consisting of a filter for microorganisms 1407 and a catalyst 1408. The purpose of the filter for microorganisms 1407, i.e. the killing-off of trapped microorganisms by continuous UV irradiation, is optimised by the arrangement of the UV tubes 1404. The air 1409 issuing from the second portion then passes into the suction fan 1410 which ensures that the air is conveyed through the purifying device. Finally, the air passes through a third portion consisting of ionisation tubes 1411. The ionisation tubes are preferably arranged perpendicularly to the direction of flow to allow a low overall height of the purifying device. The purified air 1412 issues through openings on the upper side of the housing 1402.

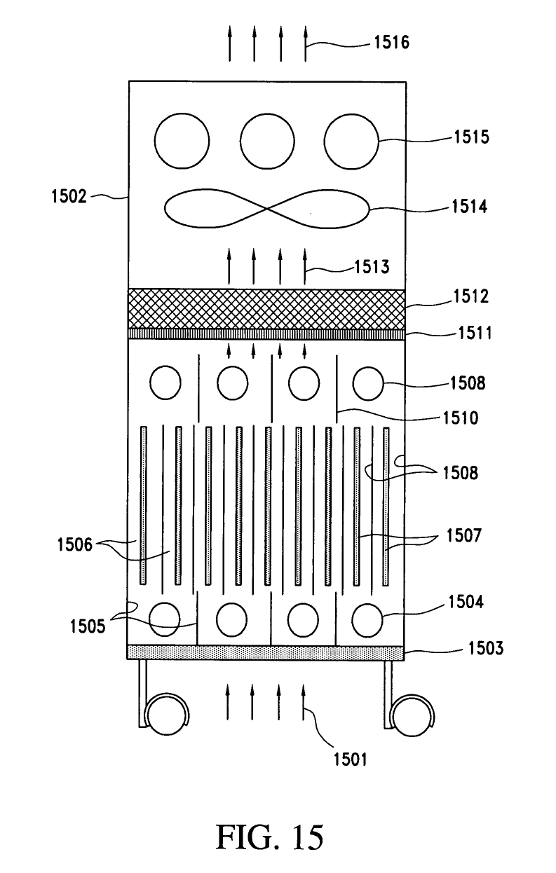

In order to ensure relatively high volume flow rates and at the same time an optimum effect of the dust and particle filters, a device according to a ninth embodiment can be used in accordance with FIG. 15.

The ambient air 1501 contaminated with pollutants passes into the purifying device via inlet openings on the underside of the housing 1502. First, the ambient air 1501 contaminated with pollutants passes through a dust filter 1503. The microorganisms trapped in this case are rendered harmless by the continuous UV irradiation of the subsequent UV tubes 1504. The UV tubes 1504 are in this case arranged perpendicularly to the direction of air flow, so optimum irradiation of the dust filter 1503 is achieved, allowing effective killing-off of trapped microorganisms. The air passed through the dust filter 1503 then passes through the first portion consisting of UV tubes 1504 and the advantageously reflective surfaces 1505. The advantageously reflective surfaces 1505, which are located between the UV tubes 1504 and also on the lateral walls of the housing 1502, intensify the effect of the UV radiation. The air then passes through a region comprising a large number of reaction channels 1506 which are arranged in a honeycomb configuration and connected in parallel. A UV tube 1507 is arranged longitudinally in each of the reaction channels 1506. The walls 1508 of the reaction channels 1506 are preferably coated with a reflective material. The arrangement of these UV tubes 1507 in the direction of flow allows the purifying device to be operated at high volume flow rates. The air then passes, again, through a region comprising UV tubes 1509 and having advantageously reflective surfaces 1510 which are arranged perpendicularly to the air flow. In addition to the primary effect of the UV radiation, for the killing-off of microorganisms located in the air, this arrangement ensures optimum irradiation of the subsequent filter for microorganisms 1511. The air pre-treated in this way then passes through the second portion consisting of a filter for microorganisms 1511 and a subsequent catalyst 1512. The air 1513 issuing from the second portion then passes into the suction fan 1514 which ensures that the air is conveyed through the purifying device. Finally, the air passes through a third portion consisting of ionisation tubes 1515. The ionisation tubes 1515 are preferably arranged perpendicularly to the direction of flow to reduce the overall height of the purifying device. The purified air 1516 issues through openings on the upper side of the housing 1502.

I claim:

1. Use of a device for breaking down gaseous hydrocarbon emissions in order to sterilise ambient air conducted in an air duct and containing microorganisms, wherein the air duct has a plurality of portions succeeding one another in the direction of flow, wherein a UV unit for irradiating the ambient air with UV radiation is provided in a first portion, the UV radiation having a first wavelength range below 240 nm for the formation of ozone and a second wavelength range above 240 nm for absorption by the microorganisms, wherein a catalyst for breaking down the ozone produced by the UV unit is provided in a subsequent second portion and wherein an ionisation unit for ionising the ambient air is provided in a subsequent third portion.

2. Use according to claim 1, wherein the first wavelength range is in the region of 185 nm and wherein the second wavelength range is in the region of 254 nm.

3. Use according to claim 1, wherein the first portion of the air duct has reflective surfaces in the region of the UV radiation.

4. Use according to claim 1, wherein the first portion of the air duct has a coating comprising a broadband semiconductor material in the region of the UV radiation.

5. Use according to claim 4, wherein the semiconductor material consists of titanium dioxide (TiO₂) or doped titanium dioxide.

6. Use according to claim 5, wherein for achieving an especially marked photocatalytic effect, a wavelength range of the UV radiation is between 350 nm and 420 nm.

7. Use according to claim 1, wherein the at least one UV emitter consists of a cylindrically configured UV lamp.

8. Use according to claim 7, wherein reaction channels arranged in a honeycomb configuration are provided parallel to the direction of flow and wherein a cylindrically configured UV lamp is arranged longitudinally in each reaction channel.

9. Use according to claim 1, wherein the catalyst is formed by catalytic activated carbon.

10. Use according to claim 1, wherein the catalyst consists of a support material formed from activated carbon, pumice stone, zeolites or clay and of an additive of catalytic metal oxides.

11. Use according to claim 10, wherein the catalyst is provided with an additive consisting of oxides of Mn, Fe, Co, Ni, Zn, Si, Ti or Zr.

12. Use according to claim 10, wherein the catalyst is provided with an additive of catalytic metal oxides in a mixture with Pt, Pd or Rh.

13. Use according to claim 1, wherein the catalyst has a thin-walled construction with low flow resistance.

14. Use according to claim 1, wherein the ionisation unit consists of at least one ionisation tube.

15. Use according to claim 1, wherein a room is supplied with ambient air via the air duct.

16. Use according to claim 15, wherein, with respect to the ambient air filling the room, the ambient air is circulated several times per hour.

17. Use according to claim 1, wherein ambient air contaminated with enveloped viruses, in particular with SARS viruses, is sterilised.

18. Use according to claim 1, wherein ambient air contaminated with enveloped viruses, in particular with avian flu viruses, is sterilised.

19. Use according to claim 1, wherein ambient air contaminated with enveloped viruses, in particular with Ebola viruses, is sterilised.

20. Use according to claim 1, wherein ambient air contaminated with enveloped viruses, in particular with influenza viruses, is sterilised.

21. Device for sterilising ambient air conducted in an air duct and containing microorganisms, wherein the air duct has a plurality of portions succeeding one another in the direction of flow, comprising a UV unit for irradiating the ambient air with UV radiation in a first portion of the air duct, the UV radiation having a first wavelength range below 240 nm for the formation of ozone and a second wavelength range above 240 nm for absorption by the microorganisms, comprising a catalyst for breaking down the ozone produced by the UV unit in a subsequent second portion, comprising an ionisation unit for ionising the ambient air in a subsequent third portion, and comprising a filter for microorganisms arranged between the first portion and the second portion.

22. Device according to claim 21, wherein the first wavelength range is in the region of 185 nm and wherein the second wavelength range is in the region of 254 nm.

23. Device according to claim 21, wherein the first portion of the air duct has reflective surfaces in the region of the UV radiation.

24. Device according to claim 21, wherein the first portion of the air duct has a coating comprising a broadband semiconductor material in the region of the UV radiation.

25. Device according to claim 24, wherein the semiconductor material consists of titanium dioxide (TiO₂) or doped titanium dioxide.

26. Device according to claim 25, wherein for achieving an especially marked photocatalytic effect, a wavelength range of the UV radiation is between 350 nm and 420 nm.

27. Device according to claim 21, wherein the at least one UV emitter consists of a cylindrically configured UV lamp.

28. Device according to claim 27, wherein reaction channels arranged in a honeycomb configuration are provided parallel to the direction of flow and wherein a cylindrically configured UV lamp is arranged longitudinally in each reaction channel.

29. Device according to claim 21, wherein a dust filter is provided before the first portion, viewed in the direction of flow.

30. Device according to claim 29, wherein cylindrically configured UV lamps for irradiating the dust filter are arranged perpendicularly to the direction of flow after the dust filter and in the region of the first portion, viewed in the direction of flow.

31. Device according to claim 21, wherein cylindrically configured UV lamps for irradiating the filter for microorganisms are arranged perpendicularly to the direction of flow before the filter for microorganisms in the region of the first portion, viewed in the direction of flow.

32. Device according to claim 21, wherein the catalyst is formed by catalytic activated carbon.

33. Device according to claim 21, wherein the catalyst consists of a support material formed from activated carbon, pumice stone, zeolites or clay and of an additive of catalytic metal oxides.

34. Device according to claim 33, wherein the catalyst is provided with an additive consisting of oxides of Mn, Fe, Co, Ni, Zn, Si, Ti or Zr.

35. Device according to claim 33, wherein the catalyst is provided with an additive consisting of catalytic metal oxides in a mixture with Pt, Pd or Rh.

36. Device according to claim 21, wherein the catalyst has a thin-walled construction with low flow resistance.

37. Device according to claim 21, wherein the ionisation unit consists of at least one ionisation tube.

38. Device according to claim 21, wherein a room is supplied with ambient air via the air duct.

39. Device according to claim 38, wherein, with respect to the ambient air filling the room, the ambient air is circulated several times per hour.

40. Device according to claim 21, wherein ambient air contaminated with enveloped viruses, in particular with SARS viruses, is sterilised.

41. Device according to claim 21, wherein ambient air contaminated with enveloped viruses, in particular with avian flu viruses, is sterilised.

42. Device according to claim 21, wherein ambient air contaminated with enveloped viruses, in particular with Ebola viruses, is sterilised.

43. Device according to claim 21, wherein ambient air contaminated with enveloped viruses, in particular with influenza viruses, is sterilised.
